(19) United States
(12) Patent Application Publication
(10) Pub. No.: US 2014/0011749 A1
(43) Pub. Date: Jan. 9, 2014

(54) KETOGENIC DIET COMPOSITION FOR THE TREATMENT OF CHEMO THERAPY AND/OR RADIATION THERAPY PATIENTS

(71) Applicant: Andrew Sean Lynch, Liverpool (GB)

(72) Inventor: Andrew Sean Lynch, Liverpool (GB)

(21) Appl. No.: 14/000,715

(22) PCT Filed: Feb. 24, 2012
(86) PCT No.: PCT/EP2012/000815

§ 371 Date: Sep. 26, 2013

(30) Foreign Application Priority Data
Feb. 24, 2011 (EP) PCT/EP2011/000905

(51) Int. Cl. A61K 38/02; A61K 31/70; A61K 31/20

(52) U.S. Cl. CPC A61K 38/02; A61K 31/20; A61K 31/70
USPC 514/19.3

(57) ABSTRACT

The present disclosure provides ketogenic compositions for use in the treatment of a cancer patient undergoing chemotherapy or radiation therapy.

### FIELD

The invention relates to a ketogenic diet composition for the treatment of chemo- and/or radiation therapy patients. The invention relates furthermore to the combination of a ketogenic diet composition with chemotherapy and/or radiotherapy for treating cancers.

### BACKGROUND

Presently oncology patients need to undergo specific treatments for their disease such as surgery, chemotherapy or radiation therapy. Although more and more successful, these therapies still need improvement.

Nutritional treatment of the side effects caused by cancer, or caused by the chemo- or radiotherapy has been suggested. It is known for a long time that oncology patients suffer from cachexia leading to increased nutritional requirements. High energy, high protein diets have been developed including all required macro and micro nutrients.

In a next step of nutritional therapy of oncology patients, ketogenic diets are recommended for slowing the growth of tumors. Ketogenic diets are diets that cause a ketogenic status in a patient. E.g. it was shown that a ketogenic diet could slow the growth of brain tumors (The calorically restricted ketogenic diet, an effective alternative therapy for malignant brain cancer. Weihua Zhou, Puma Mukherjee, Michael A Kiebish, William T Markis, John G Mantis and Thomas N Seyfried; Nutrition & Metabolism 2007, 4:5-). It is suggested that KetoCal®, a ketogenic diet formulation, is effective for the long-term management of malignant brain cancer and other glycolysis dependent cancers. This is especially pertinent to patients with glioblastoma multiforme, an aggressive brain tumor type for which few effective therapeutic options are available.

Still there is a need for improvements in the treatment of many oncology patients.

Thus, the technical problem underlying the present invention is to provide compositions, methods and means for overcoming the disadvantages in the treatment of cancers, especially of glycolysis dependent cancers.

### SUMMARY OF THE INVENTION

The inventors surprisingly found that a combination of chemotherapy or radiotherapy with a ketogenic diet can cure brain tumors in a model using mice with malignant glioma cells. The experiments were done in mice that received whole brain radiation in combination with at libitum standard feed or ketogenic diet. Irradiated animals fed standard feed had a median survival of 44.5 days, while irradiated animals maintained on ketogenic diet demonstrated a complete loss of bioluminescence, suggesting the absence of viable tumor cells. These animals have been followed for 170 days with no visible signs of recurrent tumor (p=0.0061), and tumor tissue does not recur even when animals were switched back to standard (non-ketogenic) diet. Animals fed ketogenic diet in combination with temozolomide (TMZ) chemotherapy had a median survival of 37 days compared to 28 days for animals treated with TMZ and maintained on SD (p=0.0043). Previous studies with only chemotherapy or radiotherapy never showed complete loss of tumor cells, or cure of brain tumor patients.

A preferred embodiment according to the present invention is therefore a ketogenic diet in combination with chemotherapy and/or radio-therapy for the treatment of invasive solid tumors (cancers). The ketogenic diet should preferably have a weight ratio fat : protein and optionally carbohydrates of at least 2:1, preferably at least 3:1 and even more preferably 4 to 5 :1, and most preferably 4:1. The product according to the present invention is preferably consumed in a daily amount of at least 5 kcal per kg body weight, more preferably in a daily amount of at least 10 kcal per kg body weight.

### DETAILED DESCRIPTION

Thus, the present invention solves its technical problem in particular by providing the teaching to use a composition comprising fat, protein and optionally carbohydrate, wherein the weight ratio of said fat to the sum of said carbohydrate and said protein is at least 2, for the treatment of a cancer patient undergoing chemotherapy or radiation therapy, wherein preferably the total calories provided by the composition are at least 5 kcal per kg body weight per day, more preferably the total calories provided by the composition are at least 10 kcal per kg body weight per day.

Particularly, the present invention relates to the medical use of a ketogenic diet composition, wherein the weight ratio of fat to the sum of protein and optionally carbohydrate is at least 2, for the treatment of a cancer patient together with the treatment by chemotherapy or radiation therapy. Thus, the present invention relates to a further medical use of a ketogenic diet composition, wherein a cancer patient is treated by administering an effective amount of a composition comprising fat, protein and optionally carbohydrate, wherein the weight ratio of said fat to the sum of said carbohydrate and said protein is at least 2, and wherein the patient is also treated by chemotherapy or radiation therapy. Preferably the effective amount of the composition is at least an amount, wherein the total calories provided by the composition are at least 5 kcal per kg body weight per day, more preferably the total calories provided by the composition are at least 10 kcal per kg body weight per day.

Thus, the present invention relates particularly to the surprising synergistically medical use of a ketogenic diet composition and chemotherapy or radiation therapy for treating cancers.

Preferably, the composition is used in an adjunctive therapy consisting of radiation therapy and/or chemotherapy with nutritional therapy using the composition as a ketogenic diet for treating cancer patients.

Accordingly, the present invention solves its technical problem in particular by providing a composition comprising fat, protein and optionally carbohydrate, wherein the weight ratio of said fat to the sum of said carbohydrate and said protein is at least 2, for use in the treatment of a cancer patient undergoing chemotherapy or radiation therapy, wherein the total calories provided by the composition are preferably at least 5 kcal per kg body weight per day, more preferably the total calories provided by the composition are preferably at least 10 kcal per kg body weight per day.

The present invention solves its technical problem also in particular by providing a composition comprising fat, protein and optionally carbohydrate, wherein the weight ratio of said fat to the sum of said carbohydrate and said protein is at least 2, for the manufacture of a pharmaceutical composition for the use in the treatment of a cancer patient undergoing chemotherapy or radiation therapy, wherein the total calories provided by the composition are preferably at least 5 kcal per kg body weight per day, more the total calories provided by the composition are preferably at least 10 kcal per kg body weight per day.

A preferred embodiment according to the present invention comprises a composition comprising fat, protein and optionally carbohydrate, wherein the weight ratio of said fat to the sum of said carbohydrate and said protein is at least 2, and the total calories provided by the composition are at least 5, preferably at least 10 kcal per kg body weight per day, for use in the treatment of a cancer patient undergoing chemotherapy and/or radiation therapy.

Alternatively, a preferred composition according to the present invention comprises fat, protein and optionally carbohydrate, wherein the weight ratio of said fat to the sum of said carbohydrate and said protein is at least 2, and the total calories provided by the composition are at least 50 percent of the daily requirement calculated by standard methods, e.g., Schofield or Harris-Benedict equations weight/day, for use in the treatment of a cancer patient undergoing chemotherapy or radiation therapy.

Preferably the composition comprises fat and protein. Preferably the composition comprises fat, protein and carbohydrate.

A ketogenic diet (KD) is a high-fat, low-carbohydrate diet that alters the body's metabolism to burn fats in preference to carbohydrates. The use of fats as a primary energy source leads to a state of ketosis and the accumulation of ketone bodies in the blood. KetoCal® (Nutricia North America, Gaithersburg, Md.) is a nutritionally complete, commercially available 4:1 (weight ratio fat:carbohydrate+protein) ketogenic formula that has been used as an effective non-pharmacologic treatment for the management of pediatric epilepsy. Diet-induced changes to brain homeostasis as a result of ketosis have shown potential as an alternative treatment for other neurological diseases including malignant gliomas.

In a preferred embodiment of the invention, the composition is KetoCal®.

In a preferred embodiment of the invention, the weight ratio of the fat to the sum of the carbohydrate and the protein in the composition is at least 3 to 1. In a preferred embodiment of the invention, the weight ratio of the fat to the sum of the carbohydrate and the protein in the composition is at least 3 to 1 and at most 5 to 1.

In a preferred embodiment of the invention, the weight ratio of the fat to the sum of the carbohydrate and the protein in the composition is at least 4 to 1 and at most 5 to 1, and more preferably around 4 to 1, and even more preferably 4 to 1.

In a preferred embodiment of the invention, the composition is used as a pharmaceutical composition. In a preferred embodiment of the invention, the composition is a pharmaceutical composition.

In a preferred embodiment of the invention, the composition is used in a liquid form. In a preferred embodiment of the invention, the composition is a liquid composition. In an alternative embodiment of the invention, the composition is a solid composition, for example in powder form.

Glioblastoma multiforme (GBM) is the most malignant primary brain tumor in adults and children. Conventional GBM therapies are considered palliative, but rarely curative. Long-term progression free survival remains low for most GBM patients even after complete surgical excision, combined with the best available treatment. Standard therapy for GBM includes surgery followed by concomitant radiation and/or chemotherapy. These procedures, however, extend median survival by only a few months beyond the no therapy option. The ketogenic composition according to the present invention is ideally suited as a non-toxic nutritional therapy in addition to the standard therapy for managing malignant brain cancer. Also other cancer types can be treated with a combination of a ketogenic nutritional composition and standard radiation and/or chemotherapy. It is well known in the art that cancer cells tend to become more dependent on glucose as a source of energy as they develop into later, more aggressive and malignant stages. In particular other glycolysis dependent cancers such as breast, endometrial, colorectal, lung, cervical, ovarian, head and neck cancers, and also cancers which have progressed to become metastatic.

In a preferred embodiment of the invention, the cancer to be treated is an invasive solid tumor. In a preferred embodiment of the invention, the cancer to be treated is a glycolysis dependent cancer. In a preferred embodiment of the invention, the composition is for use in the treatment of a cancer patient having a brain tumor, prostate cancer, breast cancer, endometrial cancer, colorectal cancer, lung cancer, cervical cancer, ovarian cancer or a head and neck cancer. In a preferred embodiment of the invention, the cancer to be treated is a brain tumor. In a preferred embodiment of the invention, the cancer to be treated is glioblastoma multiforme.

In a preferred embodiment of the invention the composition comprising fat, protein and optionally carbohydrate, wherein the weight ratio of said fat to the sum of said carbohydrate and said protein is at least 2, is used in the treatment of a cancer patient undergoing radiation therapy, wherein the total calories provided by the composition are preferably at least 5 kcal per kg body weight per day, more preferably the total calories provided by the composition are preferably at least 10 kcal per kg body weight per day.

In a preferred embodiment of the invention the composition comprising fat, protein and optionally carbohydrate, wherein the weight ratio of said fat to the sum of said carbohydrate and said protein is at least 2, is used in the treatment of a cancer patient undergoing chemotherapy, wherein the total calories provided by the composition are preferably at least 5 kcal per kg body weight per day more preferably the total calories provided by the composition are preferably at least 10 kcal per kg body weight per day.

Examples of chemotherapy are known to the person skilled in the art as are the treatment regimens of radiation therapy. The chemotherapy can be for example an adjuvant or a neoadjuvant chemotherapy. The chemotherapy can be for example chemotherapy using temozolomide. The chemotherapy is preferably an adjuvant chemotherapy. The radiation therapy is preferably an adjuvant radiation therapy.

In a preferred embodiment the composition is used in the treatment of a cancer patient before, during and/or after chemotherapy or radiation therapy. In a preferred embodiment the composition is used in the treatment of a cancer patient during and/or after chemotherapy or radiation therapy. In a preferred embodiment the composition is used in the treatment of a cancer patient before, during chemotherapy or radiation therapy.

Nutritional Compositions:

Enteral nutrition is giving the patient nutrients in liquid form (formula) orally or through a tube that is placed into the stomach or small intestine. The following types of feeding tubes may be used: A nasogastric tube is inserted through the nose and down the throat into the stomach or small intestine. This kind of tube is used when enteral nutrition is only needed for a few weeks. A gastrostomy tube is inserted into the stomach or a jejunostomy tube is inserted into the small intestine through an opening made on the outside of the abdomen. This kind of tube is usually used for long-term enteral feeding or for patients who cannot use a tube in the nose and throat. Formula may be given through the tube as a constant drip (continuous feeding) or 1 to 2 cups of formula can be given 3 to 6 times a day (bolus feeding). Tube feeding is sometimes used when the patient is able to eat small amounts by mouth, but cannot eat enough. Nutrients given through a tube feeding add the calories and nutrients needed for health.

Preferably the composition according to the present invention is an enteral nutrition composition.

Preferably the composition according to the present invention can be used for tube feeding. Therefore in a preferred embodiment the composition is a liquid composition with a viscosity below 70, preferably of at least 1 and at most 40, more preferably between 2 to 30 MPa·s at 100 sec⁻¹ shear rate, preferably measured at 20 degrees Celsius.

Severe weight loss and in particular muscle wasting is a serious phenomenon that occurs on a broad scale in patients suffering from diseases, disorders and trauma. Muscle wasting (MW) in chronic disease is defined as an involuntary loss of body weight of more than 5% within one month. If loss of lean body mass occurs at a more gradual rate but during a longer period, the inventors refer to chronic muscle wasting (CMW), in particular if more than 10% of body weight is lost in a period of 6 months. MW is typically observed during recovery of trauma or surgery. CMW is typically observed in severe diseases such as cancer. The rate of muscle wasting is associated with increased morbidity and mortality. The cause of muscle wasting as a result of a disease is thought to be multi-factorial. An adequate daily calorie intake is therefore very important as is a sufficient amount of micro nutrients such as vitamins and minerals.

Preferably the product according to the present invention provides a daily calorie intake of at least 5 kcal per kg body weight, more preferably of at least 5 and at most 30 kcal per kg body weight, and even more preferably between 5 and 25 kcal per kg body weight.

Preferably the product according to the present invention provides a daily calorie intake of at least 10 kcal per kg body weight, more preferably of at least 10 and at most 30 kcal per kg body weight, and even more preferably between 15 and 25 kcal per kg body weight.

The product according to the present invention is preferably consumed in a daily amount of at least 610 kcal, more preferably in a daily amount of at least 650 kcal, even more preferably of at least 700 kcal.

Preferably the product according to the present invention provides a daily calorie intake of at least 610 kcal, more preferably of at least 650 kcal, and even more preferably of at least 700 kcal.

In a preferred embodiment of the invention the product according to the present invention is preferably consumed in a non-restricted daily amount. Preferably the product according to the present invention provides a non-restricted daily calorie intake.

Fat

Fat is the main ingredient in the composition according to the present invention. A good quality of the fat is therefore essential for successful treatment and compliance. Ketones can be used as fuel by the brain and other cell types. Medium Chain Triglycerides (MCT) are medium-chain (6 to 12 carbons) fatty acid esters of glycerol, and have an improved effect on the formation of ketones and are therefore preferred fat in the ketogenic diet according to the present invention.

MCTs readily cross the mitochondrial membrane and are rapidly oxidized (beta-oxidation). This is (in part) due to the fact that fatty acids containing 6 to 12 carbons do not require carnitine to cross the mitochondrial membrane in liver tissue of healthy well-nourished adults, which is in contrast to the carnitine-dependent beta-oxidation of fatty acids with 14 carbon atoms or more. Beta-oxidation of fatty acids results in the production of acetyl-CoA that enters the Kreb's cycle to produce energy, but acetyl-CoA can also be converted into to acetoacetate, beta-hydroxybutyrate, and acetone, collectively called ketone bodies.

Therefore, the fat in the composition according to the present invention preferably comprises MCT. The fat in the composition according to the present invention preferably comprises at least 15 wt % MCT, more preferably between 15 and 95 wt % MCT and even more preferably between 15 and 60 wt % MCT based on the total fat content of the composition.

Coconut Oil is the Preferred Source of MCT.

Preferably the fat in the composition according to the present invention comprises at least 25 wt % MCT, more preferably between 25 and 65 wt % MCT and even more preferably between 35 and 60 wt % MCT based on the total fat content of the composition.

Inflammatory responses often occur in cancer patients resulting in cachexia induced weight loss. In order to improve the anti-inflammatory properties of the composition, the fat preferably contains at least one omega 3 poly unsaturated fatty acid selected from Eicosapentaenoic acid (EPA) or Docosapentaenoic acid (DHA). Accordingly the fat comprises preferably EPA and/or DHA.

According to a preferred embodiment, the following sources can be present as fat in the composition: Canola oil, high oleic sunflower oil, fish oil and/or MCT oil.

The fat contained in the composition according to the invention may be manufactured by a skilled person by combining the appropriate fat sources in appropriate amounts. According to a preferred embodiment, the following sources can be combined: Canola oil, high oleic sunflower oil, fish oil and/or MCT oil. More in particular the fat can be combined: about 37 weight % of Canola oil, about 42 weight % of high oleic sunflower oil, about 2 weight % of fish oil and about 17 weight % of MCT oil. It will be obvious to the skilled person that the amounts given above may vary to a certain extent depending on the specific composition of the fat source.

Protein and Digestible Carbohydrates

Inclusion of sufficient protein is very important to support the maintenance of lean body mass. Preferably the composition according to the present invention comprises at least 1 g protein per 100 kcal, more preferably between 1 and 4 g protein per 100 kcal, even more preferably between 1.5 and 2.5 g protein per 100 kcal.

The protein as present in the composition according to the invention, preferably comprises Leucine for at least 10 wt %, more preferably at least 12 wt %, at least 13 wt %, at least 16 wt % or at least 19 wt %, based on total protein in the composition. Usually the leucine content is 50 wt % or less, In particular, it may be 30 wt % or less, 25 wt % or less or 23 wt % or less, based on total protein. In an preferred embodiment, the leucine content is 12 to 23 wt %, based on total protein.

Total protein content preferably is selected such that the weight ratio fat to protein is at least 2:1, preferably at least 2.5:1, more preferably at least 3:1, and even more preferably at least 3:1 and at most 7.5:1, more preferably more than 3:1 and at most 7.5.1.

Optionally digestible carbohydrates can be present in the composition according to the present invention. Preferably digestible carbohydrates are present in low amounts in order to keep the ketogenic effect of the composition as high as possible. If present in the composition the amount of digestible carbohydrates is preferably lower than 10 wt % based on the total weight of the composition, more preferably below 7 wt %, even more preferably of at least 1 wt % and at most 5 wt % based on the total weight of the composition.

Indigestible Carbohydrates (Dietary Fibers)

Dietary fibers are for a large extend fermented in the colon by the bacterial flora. The fermentation process leads to the formation of short chain fatty acids (SCFA) that are used as fuel by intestinal cells, but more importantly the SCFA can contribute to the ketogenicity of the nutritional composition. A preferred embodiment according to the present invention thus includes indigestible fermentable carbon hydrates in the composition.

In particular galacto-oligosaccharides (GOS) are very suitable for manufacturing effective nutritional or pharmaceutical compositions. Suitable galacto-oligosaccharides are commercially available and include oligosaccharides—manufactured from lactose by means of a reaction with P-galactosidase. Other parts of the soluble fibre fraction can include non-oligosaccharides, like soluble and fermentable and non-fermentable fibres, including polysaccharides from the furanose type. Examples are inulin, other fructo-polysaccharides (fructans), moderately hydrolysed pectin and other gums, like glucomannans (e.g. Konjic), glucomannans (e.g. 25 guar), xanthan, and Arabic gum. These polysaccharides (having a chain length (DP) of more than 10 units) can be included up to 70 wt % of the soluble fibre composition. However, it is preferred that the fibre composition contains at least 50% wt %, more preferably at least 70% wt %, up to e.g. 95 wt %or even 98% wt % or 100% wt % of the oligosaccharides, especially the (galacto-, manno-, xylo-)oligosaccharides as defined above. The remainder may be constituted by one or more of the soluble polysaccharide fibre, especially of the fructan type. In addition to the soluble fibre fraction, non-soluble fibres can be incorporated in a fibre blend, like resistant starch, and non-soluble fermentable and non-fermentable fibres, such as cellulose. It is preferred that the non-soluble fibres represent less than 50 wt % of the soluble fibre fraction, in particular between 5 and 25 wt % thereof.

In a preferred embodiment the composition comprises dietary fibers in a range of at least 250 mg to at most 3000 mg, preferably of at least 750 mg to at most 2300 mg, more preferably of at least 1000 mg to at most 2000 mg per 100kcal product.

Adjunctive Therapy for Treating Oncology Patients

The present inventors found that adjunctive therapy consisting of traditional radiation therapy and/or chemotherapy with nutritional therapy using a ketogenic diet is particularly effective for treating cancer patients.

The present invention solves its technical problem in particular by a method for treating a cancer patient undergoing chemotherapy or radiation therapy comprising the administration of a composition comprising fat, protein and optionally carbohydrate, wherein the weight ratio of said fat to the sum of said carbohydrate and said protein is at least 2 to 1, preferably 3 to 1 and even more preferably between 2.5 to 1 and 4.5 to 1.

A preferred treatment comprises a method for treating a cancer patient undergoing chemotherapy and/or radiation therapy comprising the administration of a composition comprising fat, protein and optionally carbohydrate, wherein the weight ratio of said fat to the sum of said carbohydrate and said protein is at least 2, preferably 3, more preferably between 2.5 and 4.5, and wherein the total calories provided by the composition are at least 5 kcal per kg body weight per day, more preferably the total calories provided by the composition are at least 10 kcal per kg body weight per day.

The present invention solves its technical problem in particular by the use of a composition comprising fat, protein and optionally carbohydrate, wherein the weight ratio of said fat to the sum of said carbohydrate and said protein is at least 2, preferably 3 and even more preferably between 2.5 and 4.5, for treating a cancer patient undergoing chemotherapy or radiation therapy. Preferably the total calories provided by the composition are at least 5 kcal per kg body weight per day, more preferably the total calories provided by the composition are at least 10 kcal per kg body weight per day.

The present invention solves its technical problem in particular by the use of a composition comprising fat, protein and optionally carbohydrate, wherein the weight ratio of said fat to the sum of said carbohydrate and said protein is at least 2, in the manufacture of a nutrition composition for the treatment of a cancer patient undergoing chemotherapy or radiation therapy, wherein the total calories provided by the composition are at least 5 kcal per kg body weight per day. Preferably the composition is a composition described herein. Preferably the total calories provided by the composition are at least 10 kcal per kg body weight per day. Preferably the weight ratio of said fat to the sum of said carbohydrate and said protein is at least 3. Preferred is the use of the composition comprising fat, protein and optionally carbohydrate in the manufacture of an enteral nutrition composition for the treatment of a cancer patient undergoing chemotherapy or radiation therapy, wherein the total calories provided by the composition are at least 5 kcal per kg body weight per day, more preferably at least 10 kcal per kg body weight per day.

Further preferred embodiments are the subject matter of the sub-claims.

The invention will be further described by way of the non-limiting examples.

### EXAMPLE 1

### Ketogenic Formulation for Use in the Treatment of a Cancer Patient Undergoing Chemotherapy or Radiation Therapy

Table 1 shows preferred examples for ketogenic formulations for use in the treatment of a cancer patient undergoing chemotherapy or radiation therapy. The formulations can be provided as powder or as solution, especially aqueous solution.

TABLE 1
Ketogenic formulations

| | Per 100 g | |
|---|---|---|
| | Powder | Per 100 mL * |
| Nutrients | | |
| Calories | 720 | 144 |
| Protein, g | 15 | 3 |
| Carbohydrate, g | 3 | 0.6 |
| Fat, g | 72 | 14.4 |
| Vitamins | | |
| Vitamin A, IU (mcg) | 1265 (380) | 253 (76) |
| Vitamin D₃, IU (mcg) | 208 (5.2) | 41.6 (1) |
| Vitamin E, IU (mg) | 11 (7.4) | 2.2 (1.5) |
| Vitamin K, mcg | 40 | 8 |
| Thiamine, mg | 0.64 | 0.13 |
| Riboflavin, mg | 0.64 | 0.13 |
| Vitamin B₆, mg | 0.64 | 0.13 |
| Vitamin B₁₂, mcg | 1.3 | 0.26 |
| Niacin, mg | 7.5 | 1.5 |
| Folic Acid, mcg | 190 | 38 |
| Pantothenic Acid, mg | 2.6 | 0.52 |
| Biotin, mcg | 12.5 | 2.5 |
| Vitamin C, mg | 60 | 12 |
| Choline, mg | 480 | 96 |
| Inositol, mg | 135 | 27 |
| Minerals | | |
| Calcium, mg | 800 | 160 |
| Phosphorus, mg | 650 | 130 |
| Magnesium, mg | 110 | 22 |
| Iron, mg | 11 | 2.2 |
| Zinc, mg | 5.8 | 1.2 |
| Manganese, mg | 1.6 | 0.32 |
| Copper, mcg | 600 | 120 |
| Iodine, mcg | 82 | 16.4 |
| Molybdenum, mcg | 26 | 5.2 |
| Chromium, mcg | 19 | 3.8 |
| Selenium, mcg | 34 | 6.8 |
| Sodium, mg | 300 | 60 |
| Potassium, mg | 1080 | 216 |
| Chloride, mg | 500 | 100 |

### EXAMPLE 2

### Liquid Ketogenic Formulation

A preferred liquid formulation was prepared for cancer patients that suffer from chronic muscle wasting.

The formula contains per 100 ml:

| | |
|---|---|
| Energy | 157 kcal |
| Protein [8.2 g casein and whey, 1.8 g Leu] | 3.1 g |
| Lipids [marine oil, vegetable] | 14.5 g |
| a. EPA | 0.56 g |
| b. DHA | 0.27 g |
| Carbohydrates | 0.6 g |
| Fiber | 2.04 g |
| a. inulin DP>20 | 0.1 g |
| b. hydrolysed inulin DP<20 | 0.08 g |
| c. GOS | 1.5 g |
| d. resistant starch | 0.05 g |
| e. cellulose | 0.31 g |

### EXAMPLE 3

### Oil Blend with MCT for Use in Composition According to the Present Invention

The fat in the composition according to the invention may be manufactured by a skilled person by combining the appropriate fat sources in appropriate amounts. For example following sources can be combined: Canola oil, high oleic sunflower oil, fish oil and MCT oil. More in particular are combined: about 37 weight % of Canola oil, about 42 weight % of high oleic sunflower oil, about 2 weight % of fish oil and about 17 weight % of MCT oil.

### EXAMPLE 4

### Combination of a Ketogenic Diet and Radiation or Chemotherapy

The efficacy of KetoCal® as an adjuvant treatment for malignant glioma in combination with radiation or temozolomide (TMZ) chemotherapy was investigated.

Materials and Methods:

GL261-luc cells expressing luciferase were stereotactically implanted into the right frontal lobe of immune-competent albino C57BL/6 mice (NCI, Frederick, Md.). Following intracranial implantation, animals were fed standard rodent chow (SD) and were randomized to treatment groups on day 3 (SD vs KetoCal® with and without radiation or TMZ) based on bioluminescent signal (IVIS® Spectrum in vivo imaging system, Caliper Life Sciences, Hopkinton, Mass.). Blood levels of β-hydroxybutyrate (βHB) and glucose were measured using a Keto-Site reflectance meter (GDS Technologies, Elkhart, Ind.) and HemoCue Glucose System (HemoCue AB, Angelholm, Sweden). βHB and glucose levels were checked one and two weeks following the initiation of diet treatment. Mice were maintained on a 12 hour light/dark cycle and KetoCal® or SD was available ad libitum.

Results:

Animals fed KetoCal® had elevated levels of βHB (p=0.0173), decreased levels of glucose (p=0.0224) and an increased median survival of approximately 6 days relative to animals on SD. To determine if KetoCal® enhanced the effect of radiation, mice received 2×4 Gy whole brain radiation in combination with SD or KetoCal®. Irradiated animals fed SD had a median survival of 44.5 days, while irradiated animals maintained on KetoCal® demonstrated a complete loss of bioluminescence, suggesting the absence of viable tumor cells. These animals have been followed for 170 days with no visible signs of recurrent tumor (p=0.0061).

Animals fed KetoCal® in combination with TMZ (5×50 mg/kg) had a median survival of 37 days compared to 28 days for animals treated with TMZ and maintained on SD (p=0.0043).

In summary, the 4:1 fat:carbohydrate+protein ratio in KetoCal® appears to provide appositive effect on the treatment of malignant glioma when used in addition to radiation or TMZ. The effect is particularly pronounced when used in combination with radiation. This suggests that cellular metabolic alterations brought about through changes in the diet may be useful for the adjunctive treatment of human malignant gliomas.

1. Composition comprising fat, protein and optionally carbohydrate, wherein the weight ratio of said fat to the sum of said carbohydrate and said protein is at least 2, for use in the treatment of a cancer patient undergoing chemotherapy or radiation therapy, wherein the total calories provided by the composition are at least 5 kcal per kg body weight per day.

2. Composition according to claim 1, wherein the total calories provided by the composition are at least 10 kcal per kg body weight per day.

3. Composition according to claim 1, wherein the weight ratio of said fat to the sum of said carbohydrate and said protein is at least 3.

4. Composition according to claim 1, comprising fat, protein and carbohydrate.

5. Composition according to claim 1, wherein the fat comprises EPA and/or DHA.

6. Composition according to claim 1, wherein the fat comprises at least 25 wt % Medium Chain Triglycerides.

7. Composition according to claim 1, wherein the composition is an enteral nutrition composition.

8. Composition according to claim 1, wherein the composition is suitable for use as a tube feed and has a viscosity below 70 at 100 sec⁻¹ shear rate at 20 degrees Celsius.

9. Composition according to claim 1, wherein the composition is for use in the treatment of a cancer patient having a brain tumor, prostate cancer, breast cancer, endometrial cancer, colorectal cancer, lung cancer, cervical cancer, ovarian cancer or a head and neck cancer.

10. Composition according to claim 1, wherein the composition is for use in the treatment of a cancer patient having glioblastoma multiforme.

11. Method for treating a cancer patient undergoing chemotherapy and/or radiation therapy comprising the administration of a composition comprising fat, protein and optionally carbohydrate, wherein the weight ratio of said fat to the sum of said carbohydrate and said protein is at least 2, and wherein the total calories provided by the composition are at least 10 kcal per kg body weight per day.

12. A method for manufacture of a nutrition composition for treatment of a cancer patient undergoing chemotherapy or radiation therapy comprising forming a composition comprising fat, protein and optionally carbohydrate, wherein the weight ratio of said fat to the sum of said carbohydrate and said protein is at least 2, and wherein the total calories provided by the composition are at least 5 kcal per kg body weight per day.

13. Method according to claim 12, wherein the total calories provided by the composition are at least 10 kcal per kg body weight per day.

14. Method according to claim 12, wherein the weight ratio of said fat to the sum of said carbohydrate and said protein is at least 3.

15. Method according to claim 12, wherein the nutrition composition is an enteral nutrition composition.

16. Composition according to claim 1, wherein the composition is suitable for use as a tube feed and has a viscosity between 1-40 at 100 sec⁻¹ shear rate at 20 degrees Celsius.

17. Composition according to claim 1, wherein the composition is suitable for use as a tube feed and has a viscosity between 2-30 MPa·s at 100 sec⁻¹ shear rate at 20 degrees Celsius.

18. Method according to claim 11, wherein the weight ratio of said fat to the sum of said carbohydrate and said protein is between 2.5 and 4.5.
